(19) United States
(12) Patent Application Publication
(10) Pub. No.: US 2005/0136135 A1
(43) Pub. Date: Jun. 23, 2005

(54) Chitosan-psyllium complexes and methods for making and using same

(76) Inventor: Susie Rockway, Grayslake, IL (US)

Correspondence Address: Bell, Boyd & Lloyd LLC, P.O. Box 1135, Chicago, IL, 60690-1135, US

(21) Appl. No.: 10/741,917

(22) Filed: Dec. 19, 2003

(51) Int. Cl. A61K 35/78; A61K 31/385; A61K 31/19; A61K 31/375

(52) U.S. Cl. 424/738; 514/55; 514/440; 514/474; 514/557

(57) ABSTRACT

A composition comprising a chitosan-psyllium complex that was prepared by a solid-state enzymatic reaction. The composition can be used to reduce plasma cholesterol levels as well as fat absorption. Thus, a variety of products and methods can be used and/or developed with the composition.

### BACKGROUND OF THE INVENTION

The present invention relates to fiber complexes. More specifically, the present invention relates to fiber complexes that can reduce fat absorption and lower blood cholesterol levels.

Chitosan is a glucosamine polymer formed by deacetylating chitin. Chitosan consists of 2-acetamido-2-deoxy-β-D-glucopyranose (GlcNAc) and 2-amino-2-deoxy-β-D-glucopyranose (GlcN) units. Chitosan is believed to interact with hydrophobic compounds such as cholesterol, triglycerides, fatty and bile acids, reducing their absorption or entry into the mucosal cells of animals and man. Part of chitosan's ability to so interact is likely related to the positive charge of the amide group of the chitosan at a pH of less than 6.5, as well as chitosan's ability to solubulize at a low pH and form a viscous gel at higher pH's.

Chitosan's capacity to bind bile acids, which consequently reduces enterohepatic recycling, provides a favorable means of reducing plasma cholesterol. In addition, chitosan forms complex salts that bind to or associate with the triglycerides, fatty acids and other sterol compounds. Via ionic binding or other associations such as entrapment of hydrophobic compounds, it appears that measurable reduction of fat absorption occurs with chitosan feeding.

The physiological property of chitosan to reduce fat absorption has been tested in animal models and clinical studies. The physical properties of chitosan's fat-binding interactions have also been supported through in vitro laboratory studies. For example, chitosan has been shown to significantly reduce body weight and total plasma cholesterol. Additionally, chitosan has also shown hypocholesterolemic effects in rat feeding studies.

Psyllium is a mucilaginous material prepared from the seed husk of plants of the Plantago genus. Psyllium is a highly branched acidic arabinoxylan. The xylan backbone has both (1→4) and (1→3) linkages. Other monosaccharides presented in psyllium include D-galactose, D-rhamnose, 4-O-methyl-D-glucuronic acid, D-galacturonic acid, and 2-O-(2-D-galactopyranosyluronic acid)-L-rhamnose. Psyllium is an excellent source of both soluble and insoluble fibers.

Psyllium has recently been recognized for a number of attributes. These attributes include its cholesterol-lowering effects, laxative activity, capacity to reduce the risk of colon cancer, treatment of gastric hypoacidity, insulin sensitivity improvement capacity, and assistance in weight control.

Of course, one of the current goals in the medical community is the reduction of plasma cholesterol levels as well as reducing dietary fat intake in the general public. In many individuals, reducing plasma cholesterol levels is of paramount importance. A variety of products have been proposed and used to reduce cholesterol. These include both pharmaceutical compositions as well as diets and other treatments and therapies. Although some of these products and/or treatments have been successful, they have not been without their critics and/or side effects. Therefore, there is still a need for products and methods for reducing cholesterol and fat absorption.

### SUMMARY OF THE INVENTION

The present invention provides chitosan-psyllium complexes and methods of preparation and use. These complexes are produced by a solid-state enzymatic procedure. The complexes can be used to reduce both fat absorption and blood cholesterol levels. The fiber complexes can be used as supplements, food additives, part of a nutritional regimen, nutriceutical, or a pharmaceutical.

To this end, the present invention provides a composition comprising a chitosan-psyllium complex that is prepared by a solid-state enzymatic reaction.

In an embodiment, the ratio of psyllium to chitosan in the complex comprises 2:1. to 1:9.

In an embodiment, the composition comprises an edible acid. The edible acid can be chosen from the group consisting of fatty acids, lipoic acid, pyruvic acid, ascorbic acid, nicotinic acid, citric acid and other food grade acids.

In an embodiment, the composition comprises excipients.

In an embodiment, the composition comprises at least one flavor agent.

In a further embodiment of the present invention, a chitosan-psyllium complex is provided that is produced by a solid-state enzymatic reaction.

In an embodiment, the complex comprises 1 to 99 weight percent chitosan.

In an embodiment, the complex comprises 99 to 1 weight percent psyllium.

In another embodiment of the present invention, a method of preparing a product is provided comprising the steps of preparing a complex by reacting chitosan and psyllium using a solid-state enzymatic reaction.

In an embodiment, the enzymes are chosen from the group consisting of Xylase, Cellulase, hemicellulase, and combinations thereof.

In an embodiment, the method comprises the step of adding to a resultant chitosan and psyllium complex an edible acid.

In an embodiment, the reaction takes place at a temperature below the denaturing temperature of the enzymes(s) including ambient temperature.

Furthermore, in an embodiment, the present invention provides a method for lowering blood cholesterol levels comprising the step of administering to an individual a therapeutically-effective amount of a composition including a chitosan-psyllium complex produced by an enzymatic reaction.

In still another embodiment of the present invention, a method of reducing fat absorption in an individual is provided comprising the steps of providing to the individual a therapeutically-effective amount of a chitosan-psyllium complex produced by an enzymatic reaction.

An advantage of the present invention is to provide an improved composition including chitosan and psyllium.

Another advantage of the present invention is to provide an improved method for producing a chitosan-containing composition.

Still further, an advantage of the present invention is to provide an improved method for providing a psyllium-containing composition.

Furthermore, an advantage of the present invention is to provide a composition that can reduce blood plasma cholesterol levels.

Still further, an advantage of the present invention is to provide a composition that can reduce fat absorption in an individual consuming same.

Furthermore, an advantage of the present invention is to provide an improved pharmaceutical.

Another advantage of the present invention is to provide an improved nutriceutical.

Moreover, an advantage of the present invention is to provide an improved food additive.

Additional features and advantages of the present invention are described in, and will be apparent from, the following Detailed Description of the Invention.

### DETAILED DESCRIPTION OF THE INVENTION

The present invention provides a chitosan-psyllium complex that is produced by a solid-state enzymatic reaction. As used herein, “solid-state enzymatic reaction” refers to an enzymatic reaction carried out with limited water activity and no apparent aqueous phase in the reaction mixture.

Preferably, the complex includes 20 to 80 weight percent chitosan and 80 to 20 weight percent psyllium.

The complex can be used in compositions to reduce fat absorption in an individual consuming same. Likewise, the complex can be used in compositions to reduce blood cholesterol levels. To this end, in use, the complex can be part of a pharmaceutical, a nutriceutical, a nutritional regimen, a food additive, or a food supplement. Of course, other compositions including the complex are possible.

Generally, the enzymatic reaction to create the complex is as follows: 20 g of chitosan and psyllium powder blends were mixed with the enzyme preparation. The mixture was kept at 20-30° C. for 2-120 hours in a 100 mL beaker covered with parafilm and aluminum foil. The solid-phase enzyme reactions were terminated by inactivating enzymes in a conventional microwave oven for 1.5 min. The resulting chitosan-psyllium complexes of the solid-state enzymatic reaction were obtained after grounding the microwaved material through a 20 mesh sieve. The fat-binding capacities of the chitosan-psyllium complexes were determined using an in vitro procedure.

The enzymatic treatment of the psyllium-chitosan blend may also be conducted at any step during chitosan processing or psyllium processing.

Psyllium and chitosan may have a synergistic effect in lowering triglyceride and cholesterol plasma levels. It is believed that the solid-state enzymatic treatment alters the molecular and/or chemical structures of psyllium. This results in a new polysaccharide molecule that is believed to impact cholesterol and fatty acid absorption. In this regard, the polysaccharide complex may differ to both original polysaccharides in their physiochemical and functional properties, as well as biological activities. Therefore, solid-state enzymatic treatment of a mixture of chitosan and psyllium is believed to result in novel fiber complexes for applications in reducing fat-absorption as fat-binding agents, hypocholesterol, and laxative activity.

By way of example and not limitation, examples of procedures for making the complexes will now be set forth.

General Procedure

20 g of chitosan and psyllium powder blends was mixed with enzyme preparation. The mixture was kept at 20-30° C. for 2-120 hours in a 100 mL beaker covered with parafilm and aluminum foil. The solid-phase enzyme reactions were terminated by inactivating enzymes in a conventional microwave oven for 1.5 min. The resulting chitosan-psyllium complexes of the solid-state enzymatic reaction were obtained after ground, microwaved material was sifted and passed through a 20 mesh sieve. The fat-binding capacities of the chitosan-psyllium complexes were determined using an in vitro procedure.

The enzymatic treatment of the psyllium-chitosan blend may also be conducted at any step during chitosan processing or psyllium processing.

Analytical Procedure for Fat-Binding Capacity Evaluation

A gravimetric procedure was used to evaluate the fat-binding capacity of the chitosan-psyllium complexes using commercial olive oil. 40±5 mg of a chitosan-psyllium complex was mixed with 2.5 mL of 0.6N HCl. Twenty g of olive oil was added in the acidic slurry and mixed by vortex. After the pH of was adjusted to ˜pH 2, the mixture was incubated with shaking at 37° C. for 2 hours. 16 mL of 10 mM PBS was then added and vortexted in the mixture. The pH of the resulting mixture was brought up to pH 7 using 1N NaOH. The mixture was incubated at 37° C. for additional 30 min, and centrifuged at 2000-2200 rpm for 20 min. The unbound oil was weighted to calculate the amount of bound oil. The fat-binding capacity was expressed as grams of oil bound by per g of chitosan-psyllium complex under the testing condition. The measurements were conducted in triplicates for each sample.

Individual Examples and Analytical Results

### EXAMPLE 1

### 2003-2

0.8 g Pentopan Mono BG (Novo North America, In) was used in the reaction. This enzyme contains 2500 FXU/g. The enzyme is a purified endo 1,4-xylanase (pentosanase). The reaction mixtures contained 2 g of psyllium and 18 g of chitosan. The reaction temperature was 20-25° C. (ambient temperature), and the enzyme was inactivated by microwave heating for 1 min.

The fat binding capacity of the chitosan-psyllium complex, sample 2003-2 from Example 1, was 431.5±7.4 g oil/g chitosan-psyllium complex.

### EXAMPLE 2

### 2003-3

4 g psyllium and 16 g chitosan were used to prepare sample 2003-3. The reaction mixture contained 0.8 g of the Pentopan Mono BG enzyme. 20-25° C. reaction was performed for 48 hours.

The fat binding capacity was 476.3±7.00 g oil/g for the chitosan-psyllium complex.

### EXAMPLE 3

### 2003-4

8 g psyllium, 12 g chitosan, and 0.8 g of the Pentopan Mono BG enzyme were contained in the reaction mixture. 20-25° C. reaction was performed for 48 hours.

The fat binding capacity was 492.00±7.86 g oil/g chitosan-psyllium complex.

### EXAMPLE 4

### 2003-5

12 g psyllium, 8 g chitosan, and 0.8 g of the Pentopan Mono BG enzyme were contained in the reaction mixture. 20-25° C. reaction was performed for 48 hours.

The fat binding capacity was 428 g oil/g chitosan-psyllium complex.

### EXAMPLE 5

### 2003-6

0.5 g of the Pentopan Mono BG enzyme was mixed with 1 g psyllium and 19 g chitosan. The reaction was conducted at 20-25° C. for 42 hours.

The fat binding capacity was 451.65±12.04 g oil/g chitosan-psyllium complex.

### EXAMPLE 6

### 2003-7

2 g psyllium, 18 g chitosan, and 0.5 g of the Pentopan Mono BG enzyme were mixed, and reacted at 20-25° C. reaction for 42 hours. The enzyme was inactivated by heating in microwave oven for 1 min.

The fat binding capacity was 471.49±3.33 g oil/g chitosan-psyllium complex.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A composition comprising a chitosan-psyllium complex that was prepared by a solid-state enzymatic reaction.

2. The composition of claim 1 wherein the psyllium-chitosan ratio is 2:1. to 9:1

3. The composition of claim 1 comprising an edible acid.

4. The composition of claim 3 wherein the edible acid is chosen from the group consisting of fatty acids, lipoic acid, pyruvic acid, ascorbic acid, nicotinic acid, and combinations thereof.

5. The composition of claim 1 wherein the composition is part of a nutritional regimen.

6. The composition of claim 1 wherein the composition is a food additive.

7. The composition of claim 1 wherein the composition is a pharmaceutical.

8. The composition of claim 1 wherein the composition is a nutriceutical.

9. The composition of claim 1 comprising excipients.

10. The composition of claim 1 comprising at least one flavor agent.

11. A chitosan-psyllium complex that is produced by a solid-state enzymatic reaction.

12. The chitosan-psyllium complex wherein the complex comprises 1 to 99 weight percent chitosan.

13. The chitosan-psyllium complex wherein the complex comprises 99 to 1 weight percent psyllium.

14. A method of preparing a product comprising the steps of preparing a complex by reacting chitosan and psyllium using a solid-state enzymatic reaction.

15. The method of claim 14 wherein the enzymes are chosen from the group consisting of xylase, cellulase, hemicellulase, and combinations thereof.

16. The method of claim 14 comprising the step of adding to a resultant chitosan and psyllium complex an edible acid.

17. The method of claim 14 wherein the reaction takes place at an ambient temperature.

18. A method for lowering blood cholesterol levels comprising the step of administering to an individual a therapeutically-effective amount of a composition including a chitosan-psyllium complex produced by an enzymatic reaction.

19. The method of claim 18 wherein the psyllium to chitosan ratio comprises 2:1 to 1:9 weight percent.

20. The method of claim 18 comprising an edible acid.

21. The method of claim 20 wherein the edible acid is chosen from the group consisting of fatty acids, lipoic acid, pyruvic acid, ascorbic acid, nicotinic acid, and combinations thereof.

22. The method of claim 18 wherein the composition is part of a nutritional regimen.

23. The method of claim 18 wherein the composition is a food additive.

24. The method of claim 18 wherein the composition is a pharmaceutical.

25. The method of claim 18 wherein the composition is a nutriceutical.

26. The method of claim 18 comprising excipients.

27. The method of claim 18 comprising at least one flavor agent.

28. A method of reducing fat absorption in an individual comprising the steps of providing to the individual a therapeutically-effective amount of a chitosan-psyllium complex produced by an enzymatic reaction.
